(19) United States
(12) Patent Application Publication
(10) Pub. No.: US 2016/0015719 A1
(43) Pub. Date: Jan. 21, 2016

(54) Method of Producing Testosterone Formulation and Testosterone Formulation Produced Thereby

(71) Applicant: HYGICA BIOTECH INC., Taipei (TW)

(72) Inventors: Chien-Hung LEE, Taipei (TW); Cheng-Chieh LIN, Taipei (TW)

(21) Appl. No.: 14/507,547

(22) Filed: Oct. 6, 2014

(30) Foreign Application Priority Data
Jul. 16, 2014 (TW) 103124383

(51) Int. Cl. A61K 31/568

(52) U.S. Cl. CPC A61K 31/568

(57) ABSTRACT

The present invention is about a method of producing testosterone formulation and the testosterone formulation produced thereby. The method of this invention comprises dissolving testosterone propionate and dibucaine HCl in alcohol, adding particular percentages of polyethylene glycol 400 and polyethylene glycol 4000, and cooling under particular speed to produce the testosterone formulation which has the advantages of moderate viscosity, easy to use and excellent particle consistency.

### BACKGROUND OF THE INVENTION

1. Technical Field

The present invention relates to an improved method of producing a formulation and, more particularly, to a method of producing a testosterone formulation and a testosterone formulation produced by the method.

2. Description of Related Art

The aging of the human body is accompanied by a reduction in hormone secretion. Consequently, symptoms of physical or mental discomfort begin to show. One notable example of such discomfort is that experienced by females during menopause. Similar though not as obvious phenomena also occur in men, including those associated with a lowered testosterone level.

With the improvement of living standards, cigars as a relatively expensive Testosterone is an androgen. It is an anabolic steroid secreted mainly by the testis and the ovary and minimally by the adrenal glands. In males, testosterone is pivotal in the development of reproductive tissues (e.g., the testis and the prostate) and in inducing the secondary sexual characteristics (e.g., by increasing the growth of muscles, bone mass, and hair). In females, testosterone plays a certain role in pubic and underarm hair development, sexuality, bone density, muscle strength, and vitality. Testosterone is essential to both males and females in terms of health, in creating a sense of happiness, and in the prevention of osteoporosis. As the secretion of testosterone decreases, various phenomena can be observed such as a decline in sexual desire, difficulty in penile erection, lack of stamina for work or outdoor sports, loss of vitality, frequent sadness, and an increase in body fat.

To alleviate the foregoing negative effects of a decrease in testosterone secretion, methods for supplementing this particular hormone have been invented, which typically involve direct injection or the use of hormone patches or testosterone ointments. The injection of long-lasting testosterone at an interval of two to four weeks is convenient but tends to create significant fluctuation in the testosterone level in blood. Hormone patches are not suitable for use where temperature is high, such as in Taiwan. Testosterone ointments are a relatively good choice by comparison.

However, commercially available testosterone ointments still have their drawbacks, including, in particular, an excessively high viscosity, low particle consistency, and proneness to oil-water separation, all of which demand improvement.

### BRIEF SUMMARY OF THE INVENTION

One aspect of the invention is to provide a method of producing a testosterone formulation, comprising the steps of: A. dissolving testosterone propionate, dibucaine HCl, and methylal in alcohol to form a first solution; B. heating polyethylene glycol 400 and polyethylene glycol 4000 to about 60 to 90° C. such that the polyethylene glycol 400 and the polyethylene glycol 4000 are dissolved and form a second solution, and stirring the second solution evenly; and C. cooling the second solution obtained from step B to 25 to 75° C. at a cooling rate lower than 0.06° C./sec, adding the cooled second solution into the first solution obtained from step A to form a third solution, stirring the third solution incessantly to facilitate emulsification, and, while the third solution keeps cooling down, stirring the third solution incessantly until the third solution is emulsified into an ointment.

Preferably, the testosterone propionate, the dibucaine HCl, the methylal, and the alcohol are mixed in a ratio by weight of about 10:9:10˜20:20˜30.

Preferably, the polyethylene glycol 400 and the polyethylene glycol 4000 are mixed in a ratio by weight of about 2.5˜4:1.

Preferably, the polyethylene glycol 400 and the polyethylene glycol 4000 are mixed in a ratio by weight of about 3.08:1.

Preferably, the step B is performed in an emulsifying machine.

Preferably, the polyethylene glycol 400 and the polyethylene glycol 4000 are heated to about 80° C. in step B, and the second solution is cooled to about 70° C. in step C.

Preferably, wherein the cooling rate in step C is 0.02˜0.05° C./sec.

Preferably, wherein the cooling rate in step C is 0.04° C./sec.

Another aspect of the invention is to provide a testosterone formulation produced by the foregoing method and having a viscosity of 50×10³˜100×10³ cps.

Preferably, the testosterone formulation of claim 9, wherein the testosterone formulation has a viscosity of 50×10³-94×10³ cps.

### DETAILED DESCRIPTION OF THE INVENTION

One of the primary objectives of the present invention is to provide a method of producing a testosterone formulation, and the method includes the following steps. Step A: Testosterone propionate, dibucaine HCl, and methylal are dissolved in alcohol to form a first solution. Step B: Polyethylene glycol 400 (PEG 400) and polyethylene glycol 4000 (PEG 4000) are heated to about 60 to 90° C. and are thereby dissolved, forming a second solution, which is evenly stirred. Step C: The second solution obtained from step B is cooled to 25 to 75° C. at a cooling rate lower than 0.06° C./sec and then added into the first solution obtained from step A to form a third solution, which is incessantly stirred to facilitate emulsification. While the third solution keeps cooling down, stirring continues until the third solution is emulsified into an ointment.

Diseases and disorders caused by a decrease in testosterone secretion fall into the category of hormone disorders and include, for example, hypogonadism, female sexual disorder, reduced sexual function, and adrenal insufficiency. The major causes of primary hypogonadism include a high iron level in blood, testicular injury, hernia repair, cancer treatment, and natural aging.

The pharmacological effects of testosterone propionate are to promote genital development in young or castrated male animals, to drive mature male animals into rut, to induce secondary sexual characteristics of male animals, and to suppress the estrous cycle of female animals such that the mammary glands do not lactate even when stimulated. Testosterone propionate can prevent uterus atrophy after spaying and, when applied to aged animals, can restore their physical strength and sexual desire, whet their appetite, and increase their body weight and vigor.

Dibucaine HCl is an amide-based local anesthetic and has such pharmacological effects as to kill pain and to relieve itching.

In the foregoing method of producing a testosterone formulation, methylal (i.e., dimethoxymethane) functions as a solubilizing agent. Methylal has found wide application in medicine, cosmetics, household goods, and so on due to its excellent physical and chemical properties, namely good solubilizing ability, a low boiling point, and compatibility with water.

In the foregoing method of producing a testosterone formulation, testosterone propionate, dibucaine HCl, methylal, and alcohol are mixed in a ratio of about 10:9:10˜20:20˜30 by weight. In the conventional methods of producing like formulations, however, only testosterone propionate, dibucaine HCl, and polyethylene glycol are mixed together such that the resulting testosterone formulations tend to suffer from oil-water separation, have an undesirable feel to the touch, and therefore are not well received on the market. The present invention improves the conventional methods by adding methylal as a solubilizing agent, and to the inventor's surprise, the addition of the solubilizing agent not only overcomes the problem of oil-water separation resulting from the conventional methods, but also provides the resulting testosterone formulation with excellent particle consistency and hence a desirable feel to the touch.

In the foregoing method of producing a testosterone formulation, polyethylene glycol 400 and polyethylene glycol 4000 serve as an excipient. Generally, excipients are used in medicine to enhance the consistency and stability of drugs while reducing irritation and malodor arising from the drugs. An ideal excipient must not be toxic, irritating, pyrogenic, antigenic or hemolytic. Nor should it be pharmacologically active, lest it interfere with the functions of the main ingredients.

In the foregoing method of producing a testosterone formulation, polyethylene glycol 400 and polyethylene glycol 4000 are preferably mixed in a ratio of about 2.5˜4:1 by weight, more preferably in a ratio of 3.08:1 by weight. If the percentage of polyethylene glycol 4000 is too high, the resulting formulation will be oily and sticky. To meet consumer needs, therefore, the percentage of polyethylene glycol 4000 should not be too high. Polyethylene glycol 400, on the other hand, has a more watery feel. If the percentage of polyethylene glycol 400 is too high, a proper viscosity cannot be obtained, either. In short, the mixing ratio by weight of polyethylene glycol 400 to polyethylene glycol 4000 should be carefully selected for optimal results.

In the foregoing method of producing a testosterone formulation, the heating in step B is preferably performed in a water bath. The heating, dissolving, and stirring in step B can also be carried out in an emulsifying machine, or emulsifier. An emulsifier is a machine which disperses and emulsifies substances by centrifugation, pressing, tearing, mixing or hitting. Some common examples of such machines are vacuum emulsifiers, high-speed dispersion emulsifiers, and high-shear emulsifiers.

In the foregoing method of producing a testosterone formulation, the cooling rate in step C is lower than 0.06° C./sec and is preferably 0.04° C./sec. The lower the cooling rate is, the more viscous the resulting testosterone formulation will be. If the viscosity is too low, the testosterone formulation will flow easily and fail to form an ointment. If the viscosity is too high, the testosterone formulation will have low extensibility and have problem being squeezed out of its container. In order to form an ointment which can be easily applied by the user, the testosterone formulation of the present invention preferably has a viscosity of 50×10³ cps or more preferably 50×10³˜95×10³ cps.

In the method of the present invention, the ratio between testosterone propionate, dibucaine HCl, polyethylene glycol 400, and polyethylene glycol 4000 is preferably 10:9:740:240 or 10:9:720:260 by weight.

The present invention also provides a testosterone formulation produced by the foregoing method.

The form of the testosterone formulation is not limited to ointment.

The viscosity of the testosterone formulation preferably ranges from 50×10³ cps to 94×10³ cps.

The testosterone formulation of the present invention may be added with a fragrant essence or other additives, provided that the potency of the testosterone formulation is not compromised.

The embodiments provided below should not be construed as overly restrictive of the present invention. A person of ordinary skill in the art may modify or alter the disclosed embodiments without departing from the spirit or scope of the present invention. All such modifications and alterations fall within the scope of the present invention.

### EXAMPLES

### Examples 1˜6

The Effect of Varying the Cooling Rate and the Ratio Between Polyethylene Glycol 400 and Polyethylene Glycol 4000 on the Ointment Base

Preparation of the base: Polyethylene glycol 400 and polyethylene glycol 4000 are precisely weighed according to the table below and then evenly mixed in a closed container. Then the container is moved to a 70° C. water bath until the mixture is completely dissolved by heating. After that, the mixture is cooled according to the cooling rate specified in the following table and stirred until completely dissolved.

| | Example 1 | Example 2 | Example 3 | Example 4 | Example 5 | Example 6 |
|---|---|---|---|---|---|---|
| PEG 400 | 780 mg | 780 mg | 740 mg | 740 mg | 720 mg | 720 mg |
| PEG 4000 | 200 mg | 200 mg | 240 mg | 240 mg | 260 mg | 260 mg |
| Cooling | 0.04° C./sec | 0.8° C./sec | 0.04° C./sec | 0.8° C./sec | 0.04° C./sec | 0.8° C./sec |
| rate | | | | | | |
| Viscosity | 15.4 ± 0.4 | 12.8 ± 0.3 | 54.8 ± 0.5 | 24.7 ± 0.2 | 93.8 ± 1.6 | 50.8 ± 0.6 |
| (mean ± SD) × | | | | | | |
| 10³ cps | | | | | | |
| Tactile | Viscous | Viscous | Moderately | Considerably | Moderately | Moderately |
| evaluation | | | viscous and | viscous | viscous and | viscous and |
| | | | easy to apply | | easy to apply | easy to apply |

Preparation of the ointment: 10 mg of testosterone propionate and 9 mg of dibucaine are mixed with the base at room temperature (27° C.) by geometric dilution.

According to the experiment results, particularly those of the examples pairs having the same PEG 400/PEG 4000 ratio (i.e., examples 1 and 2, examples 3 and 4, and examples 5 and 6), the lower the cooling rate, the higher the viscosity of the formulation. In addition, the viscosities of the formulations vary with the ratio between polyethylene glycol 400 and polyethylene glycol 4000. More specifically, when the same cooling rate is used (e.g., in examples 1, 3, and 5 or examples 2, 4, and 6), the viscosities of the formulations increase with the percentage of polyethylene glycol 4000.

### Example 7

The Effect of Methylal (Serving as a Solubilizing Agent) on Viscosity

10 mg of testosterone propionate, 9 mg of dibucaine, and 16.7 ml of methylal are dissolved in 26.6 ml of alcohol. Polyethylene glycol 400 and polyethylene glycol 4000 are precisely weighed according to the table below, evenly mixed at 80° C., and cooled at a cooling rate of 0.04° C./sec. Once the temperature falls to 70° C., the polyethylene glycol mixture is added into the alcohol solution and stirred until emulsified into an ointment.

| | Example 4 | Example 7 |
|---|---|---|
| Testosterone propionate | 10 mg | 10 mg |
| Dibucaine | 9 mg | 9 mg |
| Polyethylene glycol 400 | 740 mg | 740 mg |
| Polyethylene glycol | 240 mg | 240 mg |
| 4000 | | |
| Solubilizing agent | none | methylal |
| Tactile evaluation | Moderately viscous and | Moderately viscous |
| | easy to apply | and easy to apply |
| Particle consistency* | Good | Excellent |

*Particle consistency is determined by sensory evaluation. More specifically, a blind test is conducted in which the ointments prepared in examples 4 and 7 are provided to 10 individuals, each of whom is requested to fill in a questionnaire for assessing particle consistency as felt during ointment application. In the questionnaire, particle consistency is divided into five grades: very poor, poor, fair, good, and excellent. The responses are averaged to produce the final results.

According to the example results, the addition of methylal as a solubilizing agent gives the formulation an “excellent” particle consistency, whose grade is higher than the “good” grade of the formulation without the solubilizing agent.

[Test 1] Stability Test

The stability test is performed under the shelf storage conditions of three different temperatures (4° C., 30° C., and 40° C.) and a constant relative humidity of 75%. Samples are taken in the 0th, 1st, 2nd, 3rd, and 6th months respectively for observation. The items of observation include color, smell, and consistency.

| | | Stability test | | | |
|---|---|---|---|---|---|
| | | 1st | 2nd | 3rd | 6th |
| | Properties | month | month | month | month |
| Example 1 | Color: | Color | Color | Color | Color |
| | whitish and | unchanged | unchanged | unchanged | unchanged |
| | transparent | | | | |
| | Texture: | | | | |
| | viscous | | | | |
| Example 2 | Color: | Color | Color | Color | Color |
| | whitish and | unchanged | unchanged | unchanged | unchanged |
| | transparent | | | | |
| | Texture: | | | | |
| | viscous | | | | |
| Example 3 | Color: | Color | Color | Color | Color |
| | whitish and | unchanged | unchanged | unchanged | unchanged |
| | transparent | | | | |
| | Texture: | | | | |
| | moderately | | | | |
| | viscous | | | | |
| Example 4 | Color: | Color | Color | Color | Color |
| | whitish and | unchanged | unchanged | unchanged | unchanged |
| | transparent | | | | |
| | Texture: | | | | |
| | viscous | | | | |
| Example 5 | Color: | Color | Color | Color | Color |
| | whitish and | unchanged | unchanged | unchanged | unchanged |
| | transparent | | | | |
| | Texture: | | | | |
| | moderately | | | | |
| | viscous | | | | |
| Example 6 | Color: | Color | Color | Color | Color |
| | whitish and | unchanged | unchanged | unchanged | unchanged |
| | transparent | | | | |
| | Texture: | | | | |
| | moderately | | | | |
| | viscous | | | | |
| Example 7 | Color: | Color | Color | Color | Color |
| | whitish and | unchanged | unchanged | unchanged | unchanged |
| | transparent | | | | |
| | Texture: | | | | |
| | moderately | | | | |
| | viscous | | | | |

Given the aforesaid temperatures, humidity, and sampling schedule, all the embodiments are stable and show no signs of oil-water separation during the stability test.

It can be known from the foregoing examples that the present invention provides a method of producing a testosterone formulation and a testosterone formulation produced by the method. By cooling the base of the testosterone formulation at a cooling rate lower than 0.06° C./sec, the formulation is rendered moderately viscous and easy to apply. By using methylal as a solubilizing agent in the testosterone formulation, the physical properties of the resulting ointment are further enhanced, not only featuring excellent particle consistency, but also free of the defects of the conventional formulae of like formulations, i.e., an overly high viscosity and tendency toward oil-water separation.

While the present invention has been described in connection with certain exemplary embodiments, it is to be understood that the invention is not limited to the disclosed embodiments, but, on the contrary, is intended to cover various modifications and equivalent arrangements included within the spirit and scope of the appended claims, and equivalents thereof.

What is claimed is:

1. A method of producing a testosterone formulation, comprising the steps of:
A. dissolving testosterone propionate, dibucaine HCl, and methylal in alcohol to form a first solution;
B. heating polyethylene glycol 400 and polyethylene glycol 4000 to about 60 to 90° C. such that the polyethylene glycol 400 and the polyethylene glycol 4000 are dissolved and form a second solution, and stirring the second solution evenly; and
C. cooling the second solution obtained from step B to 25 to 75° C. at a cooling rate lower than 0.06° C./sec, adding the cooled second solution into the first solution obtained from step A to form a third solution, stirring the third solution incessantly to facilitate emulsification, and, while the third solution keeps cooling down, stirring the third solution incessantly until the third solution is emulsified into an ointment.

2. The method of claim 1, wherein the testosterone propionate, the dibucaine HCl, the methylal, and the alcohol are mixed in a ratio by weight of about 10:9:10˜20:20˜30.

3. The method of claim 1, wherein the polyethylene glycol 400 and the polyethylene glycol 4000 are mixed in a ratio by weight of about 2.5˜4:1.

4. The method of claim 3, wherein the polyethylene glycol 400 and the polyethylene glycol 4000 are mixed in a ratio by weight of about 3.08:1.

5. The method of claim 1, wherein step B is performed in an emulsifying machine.

6. The method of claim 1, wherein the polyethylene glycol 400 and the polyethylene glycol 4000 are heated to about 80° C. in step B, and the second solution is cooled to about 70° C. in step C.

7. The method of claim 1, wherein the cooling rate in step C is 0.02˜0.05° C./sec.

8. The method of claim 7, wherein the cooling rate in step C is 0.04° C./sec.

9. A testosterone formulation produced by the method of claim 1 and having a viscosity of 50×10³18 100×10³ cps.

10. The testosterone formulation of claim 9, wherein the testosterone formulation has a viscosity of 50×10³˜94×10³ cps.
